(19) United States
(12) Patent Application Publication
(10) Pub. No.: US 2021/0386972 A1
(43) Pub. Date: Dec. 16, 2021

(54) MEDICAL APPARATUS WITH REFLOW TRAPPED ANCHORS AND METHOD OF USE THEREOF

(71) Applicant: Canon U.S.A., Inc., Melville, NY (US)

(72) Inventors: Showna Hsu-Hwa Chang, Arlington, MA (US); Takahisa Kato, Brookline, MA (US)

(21) Appl. No.: 17/286,397

(22) PCT Filed: Oct. 23, 2019
(86) PCT No.: PCT/US19/57711

§ 371 Date: Apr. 16, 2021

Related U.S. Application Data
(60) Provisional application No. 62/750,641, filed on Oct. 25, 2018.

(51) Int. Cl. A61M 25/01; A61B 1/00

(52) U.S. Cl. CPC A61M 25/0147; A61B 1/00154; A61B 10/04; A61M 2025/0166; A61M 2025/015

(57) ABSTRACT

An articulated medical device having a hollow core, wherein the device is capable of maneuvering through cavities to reach a target with minimal invasiveness, and once the medical device has reached the target, allowing a medical tool to be guided through the hollow cavity for facilitating medical procedures, including endoscopes, cameras, and catheters, at the target.

### CROSS REFERENCE TO RELATED PATENT APPLICATIONS

This application claims priority to U.S Provisional Patent Application No. 62/750,641, titled “Medical Apparatus with Reflow Trapped Anchors and Method of Use Thereof” filed on Oct. 25, 2018, the disclosure of which is herein incorporated in its entirety by reference.

### FIELD OF THE DISCLOSURE

The present disclosure relates generally to apparatus and methods for medical application. More particularly, the subject disclosure is directed to an articulated medical device having a hollow cavity, wherein the device is capable of maneuvering within a patient, and allowing a medical tool to be guided through the hollow cavity for medical procedures, including endoscopes, cameras, and catheters.

### BACKGROUND OF THE DISCLOSURE

Bendable medical instruments such as endoscopic surgical instruments and catheters are well known and continue to gain acceptance in the medical field. The bendable medical instrument generally includes a flexible body commonly referred to as a sleeves or sheaths. One or more tool channels extend along (typically inside) the flexible body to allow access to a target located at a distal end of the body.

The instrument is intended to provide flexible access within a patient, with at least one curve or more leading to the intended target, while retaining torsional and longitudinal rigidity so that a physician can control the tool located at the distal end of the medical instrument by maneuvering the proximal end of the medical instrument.

Recently, to enhance maneuverability of the distal end of the instrument, robotized instruments that control distal portions have emerged. In those robotized instruments, to create curves locally at the distal portion by robotics, different techniques have been disclosed.

By way of example, United States patent publication number 2016/0067450, provides multiple conduits to retain the shape of the proximal part, while the driving tendons are bending the distal part in the medical instruments. The multiple conduits would be controlled selectively in a binary way by constraining or unconstraining the proximal ends of the conduits. By selecting the constrained conduits, the bendable medical device can change the length of bending distal segment by changing the stiffness of the bendable medical device based on the area where the conduits deploy.

However, there remains a need in the industry to further refine and advance bendable medical devices to minimize the outer size (diameter) of the bendable medical instrument, and to maximize the size (diameter) of the tool channel allowing for larger/more effective tools.

### SUMMARY

Thus, to address such exemplary needs in the industry, the presently disclosed apparatus teaches a medical apparatus comprising: a bendable body comprising: a hollow cavity extending the length of the bendable body; and a wall formed about the hollow cavity; at least one control wire slideably situated in the wall; and an anchor configured at a distal end of the at least one control wire, wherein, the anchor is affixed within the wall.

In various embodiments, the anchor may extend along an axial direction of the control wire with at least one projection element configured for embedding into the wall, thus embedding the anchor into the wall of the apparatus.

In another embodiment, the wall comprises at least one lumen configured to allow the control wire to slideably rest in the wall.

In additional embodiments, a second control wire may be slideably situated in the wall, having its own anchor for affixing the second control wire to the wall, wherein the position of the anchor for the first and the second control wires are different along the axial direction of the bendable body. In addition, additional control wires may be added to the apparatus.

In various other embodiments, the at least one control wire may includes a plurality of anchors, wherein the position of each anchor is different along the axial direction of the bendable body.

In another embodiment, the medical apparatus further comprising a driving unit in communication with the at least one control wire, configured to actuate the at least one control wire in the wall.

In yet another embodiment, the at least one control wire may further comprise of an outer wire and an inner wire, wherein the inner wire is slideably nested within the outer wire. In addition, each of the inner wire and outer wire may have one or more anchors for affixing the outer wire to the wall and the inner wire has an anchor for affixing the inner wire to the wall, wherein the position of the anchor for the outer wire and the inner wires are different along the axial direction of the bendable body.

In further embodiments, the control wire and/or anchor may comprise of a radio opaque material.

In additional embodiments, a functional probe may be selected from the group consisting of a position tracking sensor, a shape sensor, an endoscopic imaging probe, anchor, control wire and combinations therefrom.

In other embodiments, the affixing of the anchor to the wall may comprise of heating the wall and anchor to create a fusion.

The subject apparatus may be used in a variety of applications and by a variety of methods, including the steps of: providing a bendable body comprising: a hollow cavity extending the length of the bendable body; a wall formed about the hollow cavity; at least one control wire slideably situated in the wall; and an anchor configured at a distal end of the at least one control wire; heating the anchor configured in the wall to create a fusion between the wall and anchor; and cooling the anchor configured in the wall to set the fusion.

In other embodiments, use of the medical apparatus may involve: providing a medical apparatus comprising: a bendable body having a hollow cavity extending the length of the bendable body, and a wall formed about the hollow cavity; at least one control wire slideably situated in the wall; and an anchor configured at a distal end of the at least one control wire wherein the anchor is affixed to the wall; advancing the medical apparatus into a subject; bending the medical apparatus to accommodate obstacles in the subject; and treating the subject once the medical apparatus advances to a desired target in the subject.

In yet additional embodiments, the medical apparatus may be amended to comprise: a bendable body having a plurality of wire guides spaced a distance from each other and extending the length of the bendable body, where a wall is formed about the plurality of wire guides; wherein at least one control wire slideably situated in at least one wire guide; and an anchor is configured at a distal end of the at least one control wire, with the anchor affixed to the at least one wire guide.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 is a block diagram of an exemplary bendable medical device incorporating various ancillary components, according to one or more embodiment of the subject apparatus, method or system.

FIG. 2a depicts a perspective close-up view of an exemplary bendable medical device, according to one or more embodiment of the subject apparatus, method or system.

FIG. 2b depicts a perspective view of an exemplary bendable medical device, according to one or more embodiment of the subject apparatus, method or system.

FIG. 3a provides a cut-away view of an exemplary bendable medical device inserted into a cavity, according to one or more embodiment of the subject apparatus, method or system.

FIG. 3b provides a perspective view of an exemplary bendable medical device depicting various orientation options, according to one or more embodiment of the subject apparatus, method or system.

FIG. 3c depicts a perspective view of an exemplary bendable medical device depicting various orientation options, according to one or more embodiment of the subject apparatus, method or system.

FIG. 4a provides a perspective view of an exemplary bendable medical device detailing an anchoring segment, according to one or more embodiment of the subject apparatus, method or system.

FIG. 4b is a detailed cross-sectional view of an exemplary bendable medical device and anchoring segment, according to one or more embodiment of the subject apparatus, method or system.

FIG. 4c provides a close-up perspective view of an exemplary bendable medical device, according to one or more embodiment of the subject apparatus, method or system.

FIG. 5 provides a side perspective view of an exemplary bendable medical device, according to one or more embodiment of the subject apparatus, method or system.

FIG. 6a depicts a side perspective view of an exemplary bendable medical device, according to one or more embodiment of the subject apparatus, method or system.

FIG. 6b depicts a side perspective view of an exemplary bendable medical device, according to one or more embodiment of the subject apparatus, method or system.

FIG. 6c depicts a side perspective view of an exemplary bendable medical device and heat transfer unit, according to one or more embodiment of the subject apparatus, method or system.

FIG. 7a illustrates various views of an exemplary bendable medical device anchor elements, according to one or more embodiment of the subject apparatus, method or system.

FIG. 7b illustrates various views of an exemplary bendable medical device anchor elements, according to one or more embodiment of the subject apparatus, method or system.

FIG. 7c illustrates various views of an exemplary bendable medical device anchor elements, according to one or more embodiment of the subject apparatus, method or system.

FIG. 8a provides a side cross-sectional view of an exemplary bendable medical device and anchoring segment, according to one or more embodiment of the subject apparatus, method or system.

FIG. 8b depicts a side cross-sectional view of an exemplary bendable medical device and anchoring segment, according to one or more embodiment of the subject apparatus, method or system.

FIG. 8c provides a side cross-sectional view of an exemplary bendable medical device and anchoring segment, according to one or more embodiment of the subject apparatus, method or system.

FIG. 9a is a side cross-sectional view of an exemplary bendable medical device, according to one or more embodiment of the subject apparatus, method or system.

FIG. 9b provides a cross-sectional view of an exemplary bendable medical device, according to one or more embodiment of the subject apparatus, method or system.

FIG. 9c illustrates a cross-sectional view of an exemplary bendable medical device, according to one or more embodiment of the subject apparatus, method or system.

FIG. 10 depicts a close-up side perspective view of an exemplary bendable medical device, according to one or more embodiment of the subject apparatus, method or system.

FIG. 11 depicts a side perspective three-dimensional view of an exemplary bendable medical device, according to one or more embodiment of the subject apparatus, method or system.

FIG. 12 depicts a side perspective three-dimensional view of an exemplary bendable medical device, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

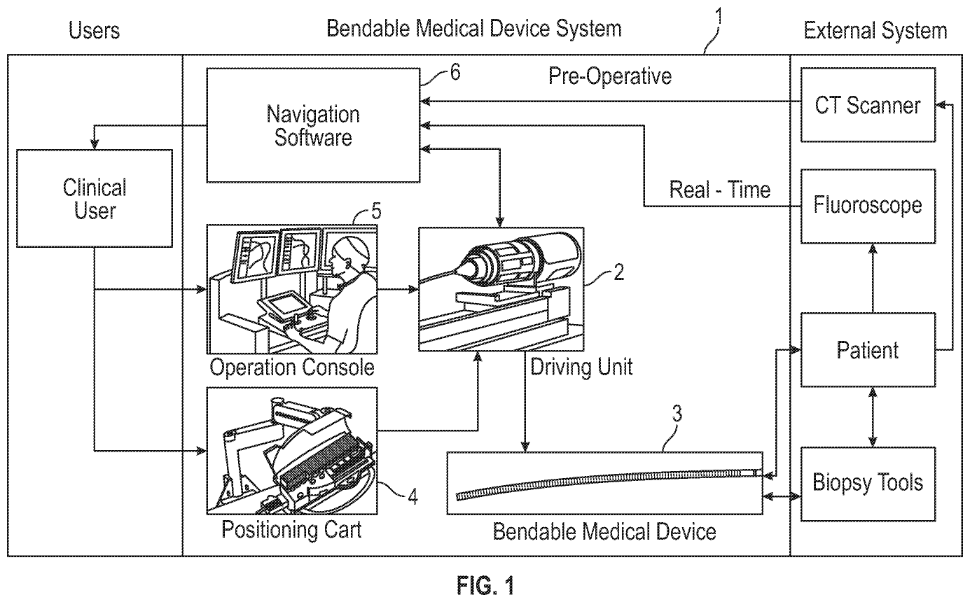

FIG. 1 is a system block diagram of an exemplary bendable medical device system 1 incorporating various ancillary components intended to amass a complete medical system. The bendable medical device system 1 comprises a driving unit 2, a bendable medical device 3, a positioning cart 4, an operation console 5 and navigation software 6. The exemplary bendable medical device system 1 is capable of interacting with external system component and clinical users to facilitate use in a patient.

The navigation software 6 and the driving unit 2 are communicatively-coupled via a bus to transmit/receive data between each other. Moreover, the navigation software 6 is connected and may communicate with a CT scanner, a fluoroscope and an image server (not in Figure), which are ancillary components of the bendable medical device system 1. The image server may include, but is not limited to, a DICOM™ server connected to a medical imaging device including but not limited to a CT and/or MRI scanner and a fluoroscope. The navigation software 6 processes data provided by the driving unit 2 and data provided by images stored on the image server, and/or images from the CT scanner and the fluoroscope in order to display images onto the image display.

The images from the CT scanner may be pre-operatively provided to navigation software 6. With navigation software, a clinical user creates an anatomical computer model from the images. In this particular embodiment, the anatomy is that of a lung with associated airways. From the chest images of the CT scanner, the clinical user can segment the lung airways for clinical treatments, such as biopsy. After generating the lung airway map, the user can also create plan to access the lesion for the biopsy. The plan includes the airways to insert and maneuver the bendable medical device 3 leading to the intended target, which in this example is a lesion.

The driving unit 2 comprises actuators and a control circuitry. The control circuitry is communicatively-coupled with operation console 5. The driving unit 2 is connected to the bendable medical device 3 so that the actuators in the driving unit 2 operate the bendable medical device 3. Therefore, a clinical user can control the bendable medical device 3 via the driving unit 2. The driving unit 2 is also physically connected to a positioning cart 4. The positioning cart 4 includes a positioning arm, and locates the driving unit 2 and the bendable medical device 3 in the intended position with respect to the target/patient. The clinical user can insert, maneuver and retreat the bendable medical device 3 to perform medical procedures, here a biopsy in the lungs of the patient.

The bendable medical device 3 can be navigated to the lesion in the airways based on the plan by the clinical user's operation. The bendable medical device 3 includes a hollow cavity for various tools (e.g. a biopsy tool). The bendable medical device 3 can guide the tool to the lesion of the patient. In one example, the clinical user can take a biopsy sample from the lesion with a biopsy tool.

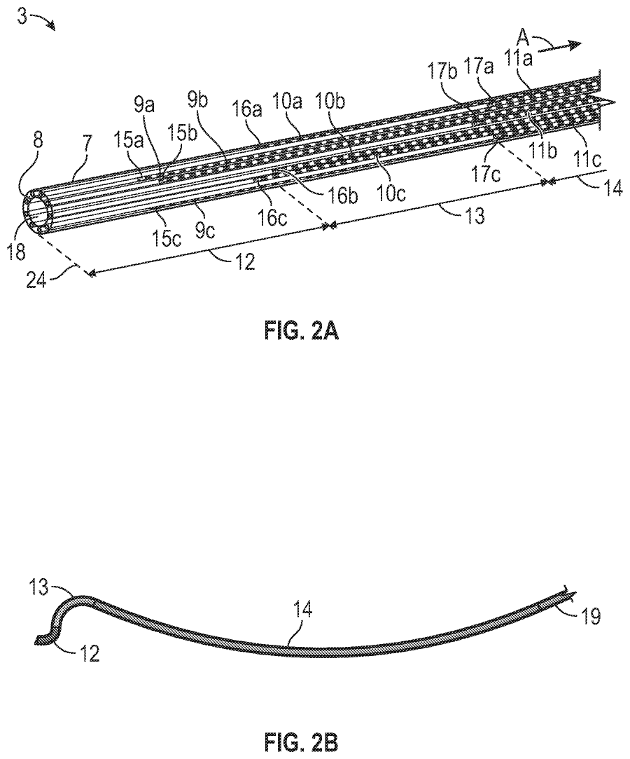

FIGS. 2a and 2b are schematic drawings of the bendable medical device 3. FIG. 2a is a perspective close-up view of the bendable medical device 3. FIG. 2b is a schematic drawing to explain the bendable segments of the bendable medical device 3. The bendable medical device 3 has a distal end 24 and a proximal end (in direction of arrow A), and comprises a proximal part 19 and three bendable segments, which are the first, second, and third bendable segments 12, 13, 14, respectively.

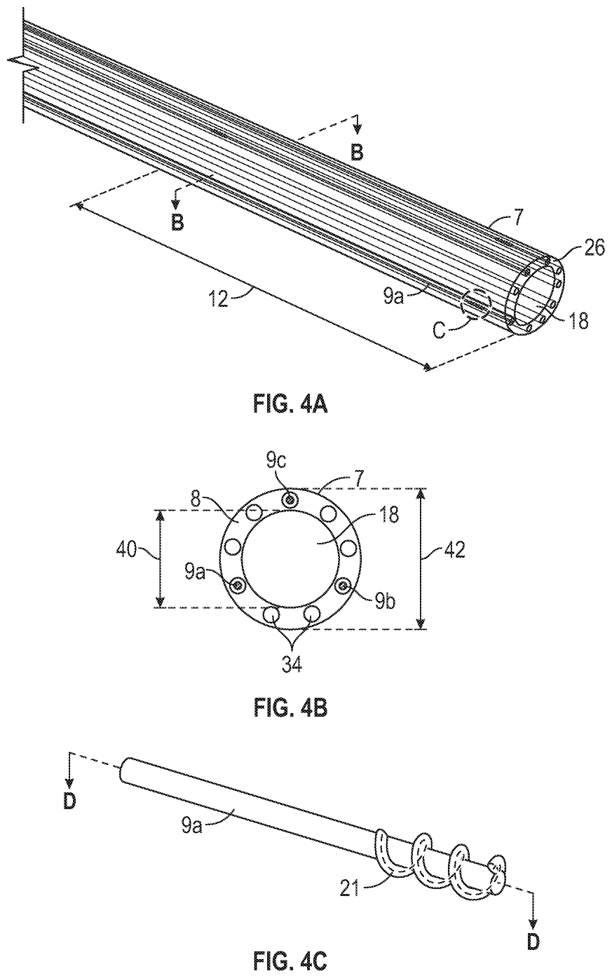

The bendable segments 12, 13, 14, can independently bend and can form a shape with three independent curvatures, as seen in FIG. 2b. The bendable medical device 3 includes a bendable body 7 with an inner diameter 40 and an outer diameter 42 (see FIG. 4B), which creates the cylindrical wall 8 of the bendable body 7, wherein the inner diameter establishes a tool channel 18 (see FIG. 4b). The wall 8 may house several lumens 34 intended to house a control wire (see FIG. 1i). The tool channel 18 is configured to extend the length of the bendable body 7, wherein the proximal part 19 of the bendable body 7 provides access to clinical users for inserting/retreating a medical tool. For example, a clinical user can insert and retrieve a biopsy tool trough the tool channel 18 to the distal end 24 of the bendable medical device 3. This may be accomplished after the bendable device 3 is inserted into the patient, or in unison with insertion/retreating the bendable device 3.

The bendable body 7 includes a set of first control wires 9a, 9b, 9c, a second set of control wires 10a, 10b, 10c, and a third set of control wires 11a, 11b, 11c. The wall 8 houses the control wires 9a-11c in the lumens 34, which are configured along longitudinal direction of the bendable body 7. The lumens 34 allow for slideable movement of the control wires 9a-11c along an axial direction of the bendable body. The control wires 9a-11c are terminated at the distal end of each bendable segment 12, 13 and 14, to form the three bendable groups, each with three wires each (a, b, c). The first control wires 9a, 9b, 9c are terminated at the distal end of the first bendable segment 12 with anchoring segments 15a, 15b, 15c, and are configured apart from each other by approximately 120 degrees within the wall 8. The first control wires 9a, 9b, 9c are connected to the driving unit 2 at the proximal end of the wires 9a, 9b, 9c. The driving unit 2 induces pushing or pulling forces to move the control wires 9a, 9b, 9c by actuating those wires, and bending the bendable body 7 from the distal end 24.

Similarly, the second set of control wires 10a, 10b, 10C are terminated at the distal end of the second bendable segment 13, using the anchoring segments 16a, 16b, 16c, and are connected to the driving unit 2 at the proximal end. The second set of control wires 10a, 10b, 10c are also housed in the wall 8. The second set of control wires 10a, 10b, 10c can bend the bendable body 7 from the distal end of the second bendable segment 13.

In the same way, the third set of control wires 11a, 11b, 11c are also configured to bend the bendable body 7 at the third bendable segment 14, once again by inducing pushing or pulling, actuated at the distal end 24 of the control wires 11a, 11b, 11c by the driving unit 2.

Accordingly, by pushing and pulling the set of control wires 9, 10, 11, the first, the second and the third bendable segments 12, 13, 14, respectively, individually bend the bendable medical device 3, in three dimensions.

The subject bendable medical device 3 incorporates control wires 9, 10, 11, that can be fixed to the bendable body 7 by using minimal space in the bendable body wall 8. Because the anchoring segment 15-17 is localized within the individual lumens 34, the bendable medical device 3 with the control wires 9, 10, 11, can be effectively miniaturized, especially when using multiple control wires 9, 10, 11. Additionally, the control wires 9, 10, 11, can be fully contained within the bendable body 7 wall 8, not needing to be outside the outer diameter 42 or inside the inner diameter 40; thus not impinging on the tool channel 18 or unnecessarily increasing the overall size of the medical device 3. By embedding the anchor segments 15-17 in the wall 8 of the bendable body 7, the control wires 9, 10, 11, can transmit pushing force, torque as well as pulling force to the bendable body 7. Therefore, the bendable medical device 3 can reduce the number of control wires 9, 10, 11, or force load per the control wire 9, 10, 11, to achieve the target bending maneuver in comparison to the conventional tendon-driven system with pulling forces.

Moreover, because the anchoring segment 15-17 does not need additional abutment parts that cover the cross section of the bendable body 7, the bendable medical device 3 can reduce the number of jointing points between the bendable body 7 and those additional abutment parts at the position of the anchoring segment 15-17. Therefore, the subject bendable medical device 3 can improve continuity of flexibility with reduced influence of motion control and reduce risks of failure in those jointing points. Also, the bendable medical device 3 can have a continuous smooth surface on the outer diameter 42 and the inner diameter 40 of the bendable body 7 to avoid risk of trauma to the patient anatomy and improved tool advancement/retraction in the tool channel 18.

Furthermore, the control wire 9, 10, 11, can be fixed to the bendable body at a wide variety of positions along the length of the bendable body 7, allowing the bendable medical device 3 to be configured to have multiple bending segments, especially a distal bending segment manipulated independently from the proximal part of the bendable body to provide improved flexible access to the intended treatment area of the patient.

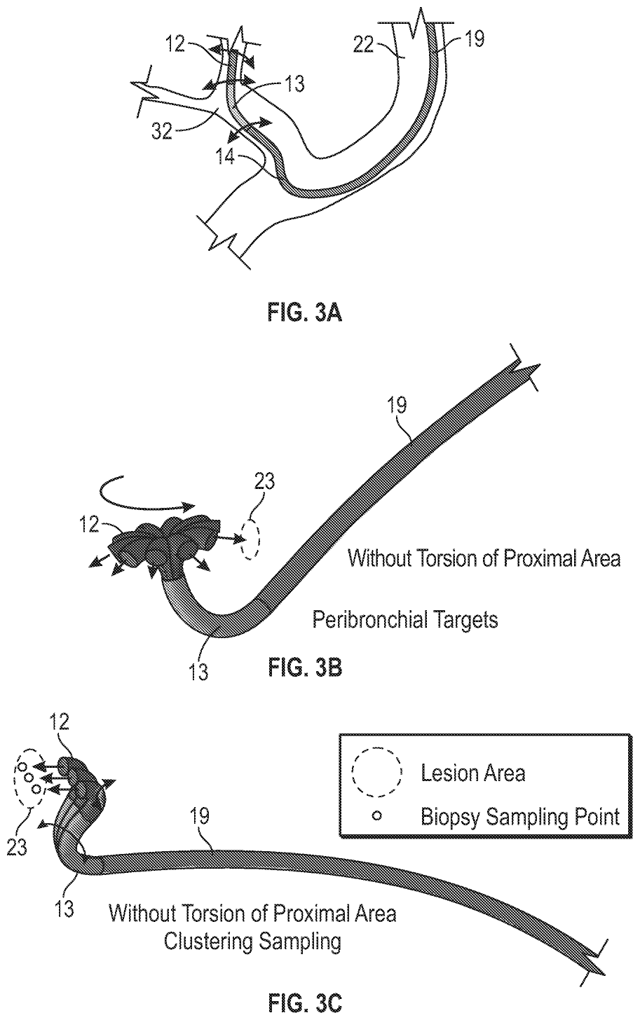

FIG. 3a provides a cut-away view of an exemplary bendable medical device 3 inserted into a cavity, wherein FIGS. 3b and 3c provide perspective views of an exemplary bendable medical device 3 depicting various orientation/maneuvering options.

FIGS. 3a, 3b and 3c are schematic drawings exemplifying the navigation and targeting of a lesion in peri-bronchial area of a patient's lungs, which is a lateral area surrounding the airways. This area is a known challenge to target as identified in literature and the prior art, due to the limited distal dexterity of the conventional catheter. To reach the lesion through airways 22 in the navigation stage, the first and the second bendable segments 12, 13, respectively, navigate the bendable medical device 3 through the bifurcation point 32. The first bendable segment 12 can adjust the shape/orientation to the daughter branch while the second bendable segment 13 can adjust the shape/orientation to the parent branch in the bifurcation point 32, as the bendable medical device 3 advances through the bifurcation point 32. Once the first and the second bendable segments 12 and 13 pass the bifurcation point 32, those segments may act as guides for the rest of the bendable medical device 3, so that the insertion force from the proximal end of the single catheter can be effectively transformed into the insertion force for a distal part of the single catheter without serious prolapsing of the distal section. Once the distal end 24 of the bendable medical device 3 reaches the vicinity of the lesion, the bendable medical device 3 would direct the distal end 24 to the lesion 23, which locates the lateral area around the airway, by bending the first and the second bendable segments 12 and 13, respectively. Since the airway doesn't directly connect with the lesion 23, this is one of the more difficult configurations for a conventional catheter.

With the first, the second and the third bendable segments 12, 13 and 14, respectively, the bendable medical device 3 can orient the distal end 24 without moving the proximal part 19 that goes through all bifurcations to this lesion. By using the three-dimensional bending capability of the first and the second bendable segments 12 and 13, the bendable medical device 3 can perform unique maneuvers to enhance capability of the peri-bronchial targeting (FIGS. 3b, 3c). Furthermore, by incorporating different positions of the anchoring segments 15-17 between the first 9, second 10 and third ii control wires along the axial direction of the bendable body 7, the bendable body 7 can function as bending objects differently along the axial direction, because the control wires 9, 10, 11, are mapped to the different position of the bendable body 7. Therefore, the bendable medical device 3 can provide improved access to the intended lesion through tortuous pathways. Also, the bendable medical device 3 can have different flexibility along the axial direction without increasing the size or number of the jointing points.

In a first maneuver in an omni-directional orientation (FIG. 3b), the first bendable segment 12 can effectively rotate without rotating any part of the bendable medical device 3. This maneuver is beneficial to determine the orientation of the distal end to the lesion 23 since this motion isn't affected by the physical interaction of the proximal part of the catheter to the anatomy, as well as not affecting the position of lesion 23, while physically mapping the orientation of the distal end 24. Moreover, with the second bendable segment 13, the bendable medical device 3 can perform this omni-directional orientation after going through the final bifurcation point to reach the lesion 23. During this rotation, the bendable medical device 3 can rotate the bending plane of only the first bendable segment 12 without moving the second and the third bendable segments 13 and 14.

The second maneuver is a clustering sampling, as provided in FIG. 3c. The first bendable segment 12 can dislocate the position of the distal end while keeping the orientation of the distal end. With this maneuver, the distal end can access the different positons in the lesion 23. The advantage of this maneuver is to access different positions in the lesion 23, or to perform fine adjustment of the position of distal end. The bendable medical device 3 can dislocate the distal end with the identical orientation. Therefore, the resolution (and accuracy/precision) of the positioning is directly related to the dislocation of the distal end.

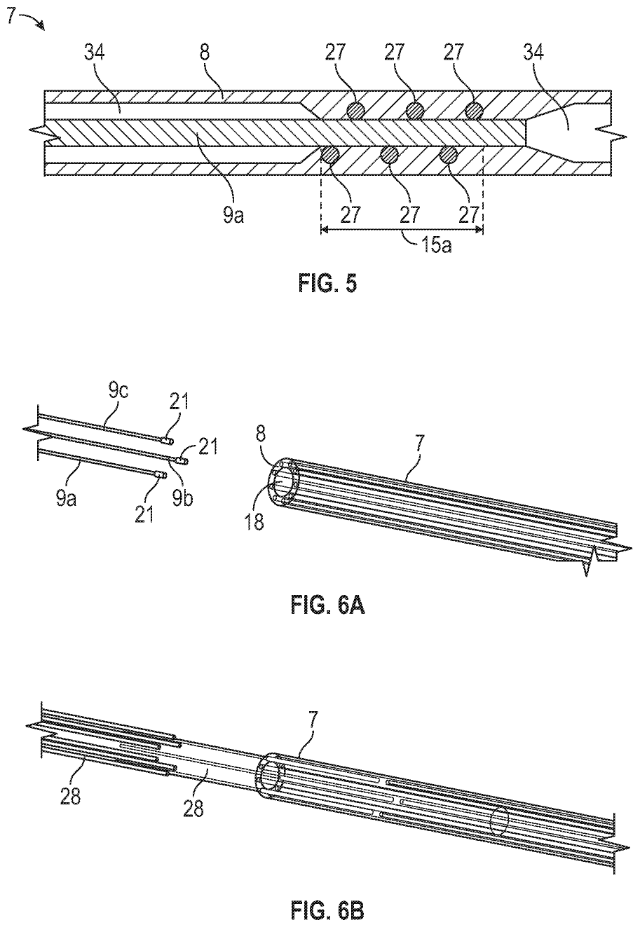

FIGS. 4a, 4b, 4c and FIG. 5 are illustrations detailing the various structures of the anchor segments 15-17. FIG. 4a is the close-up view of the distal end 24 of the bendable medical device 3. In this particular embodiment, the bendable body 7 is a tube made of polyether block amide, and has an outer diameter of 3 mm and an inner diameter of 2 mm for the tool channel 18. The edge of the bendable body 7 is rounded to create an atraumatic tip 26. FIG. 4b is a cross sectional view of line B-B in FIG. 4a. The bendable body 7 includes nine lumens 34 in the wall 8, configured to partially house the control wires, 9a, 9b, 9c which, in this embodiment, have a diameter of 150 microns, each. The first control wires 9a, 9b, 9c run through the wall 8 of bending body 7 and terminate at the distal end of the first bendable segment 12.

FIG. 4c and FIG. 5 are a close-up view of box C in FIG. 4a. Specifically, FIG. 5 is a cross sectional view at line D-D in FIG. 4c. The first control wire 9a includes a spiral coil structure as an anchor element 21. The anchor element 21 is affixed on the first control wire 9a. This affixing can be performed with welding, brazing and/or adhesive. After affixing, the spiral coil structures become projection elements 27, which are embedded into the wall 8. By using a spiral coil structure as the anchor element, accuracy of height and pitch of the projection elements 21 in a miniature size can be controlled. Also, it is advantageous to have a self-clamping feature by leveraging the restoration force of spring in the assembly process for the control wire and the anchor element. Moreover, these projection elements 27 along the longitudinal direction of the wire can be created with only one part. The projection elements 27 are embedded into the wall 8 (FIG. 5). Because the projection elements deploy along the longitudinal length of the control wire, the first anchoring segment 15a is also formed along the first control wire 9a. To increase connection and/or attachment strength, the length and/or height of the projection of the anchoring segment 15a can be increased. In the first anchoring segment 15a, the first control wire 9a is constrained by the bendable body 7. Therefore, the first control wire 9a can transmit pushing, pulling forces and torque to the bendable body 7 to actuate the first bendable segment 12. As can be appreciated, the remaining control wires 10a-11c share common properties and configuration similarities with control wires 9a-9c.

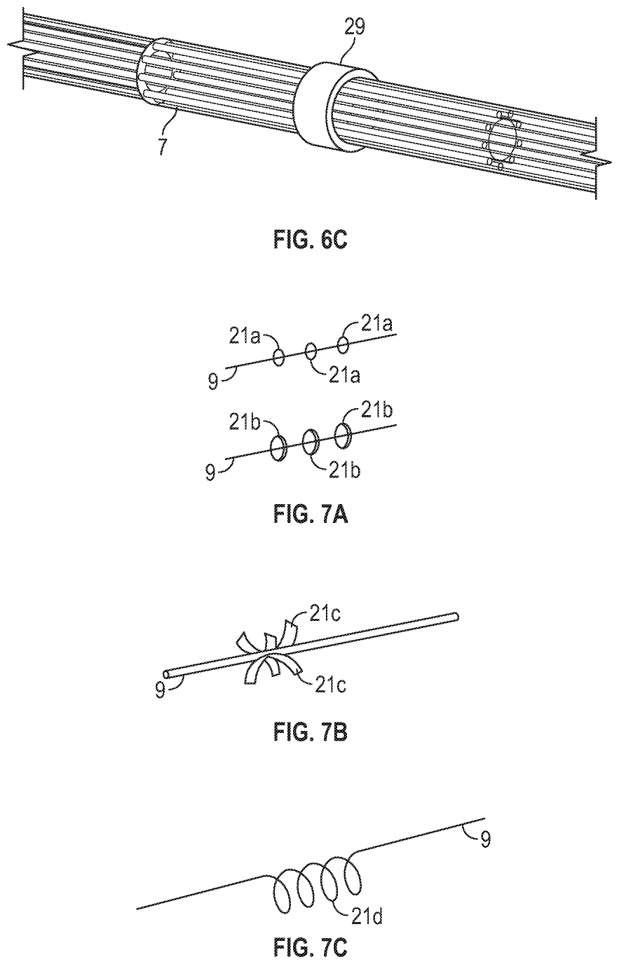

FIGS. 6a, 6b, 6c explain a fabrication process for creating the bending body 7 and imbedding the anchor segments 15-17. First, the first control wires 9a, b, c and anchor elements 21 are assembled to have anchor elements 21 at the designated positions in the first control wires 9a, 9b, 9c (FIG. 6a). The assembled first control wires 9a, 9b, 9c with the anchor elements 21 are inserted into the appropriate lumens 34 of the wall 8 at a desired position. In the remaining uninhabited lumens 34, mandrels 28 may be inserted. Also, a larger mandrel 28 may be inserted into the tool channel 18 (FIG. 6b). The mandrels 28 are inserted to retain the structural integrity and dimensions of the uninhabited lumens 34 and tool channel 18. With a heater 29, the bendable body 7 is warmed/reflowed only around the anchor element 21 to embed the projection elements 27 into the wall 8 of the bendable body 7 (FIG. 6c). The heating process creates a fusion between the anchor elements 21 and bendable body 7/wall 8, wherein the fusion may be accomplished by a physical process that results in the phase transition of one or more substance from a solid to a liquid, followed by allowing the one or more substance to cool, thus returning the fused one or more substance back to a solid. Through those steps, the anchor elements 21 are embedded by using industry-standard catheter manufacturing steps.

FIGS. 7a, 7b, 7c depict various embodiments of the anchor elements 21. One embodiment employs beads and disks as the anchor element 21a, 21b. With the beads and the disks, the anchoring segments 15-17 can be created with different lengths from the identical unit parts. Other embodiments can include leaf springs as the anchor element 21c. With the leaf springs, both the height of the projection element and the attachment strength between the bendable body 7 and the anchoring element 21c can be increased. Another embodiment may employ a spiral coil feature in the first control wire 9. With this feature, the number of parts, the number of bonding points and assembly steps/time can be reduced.

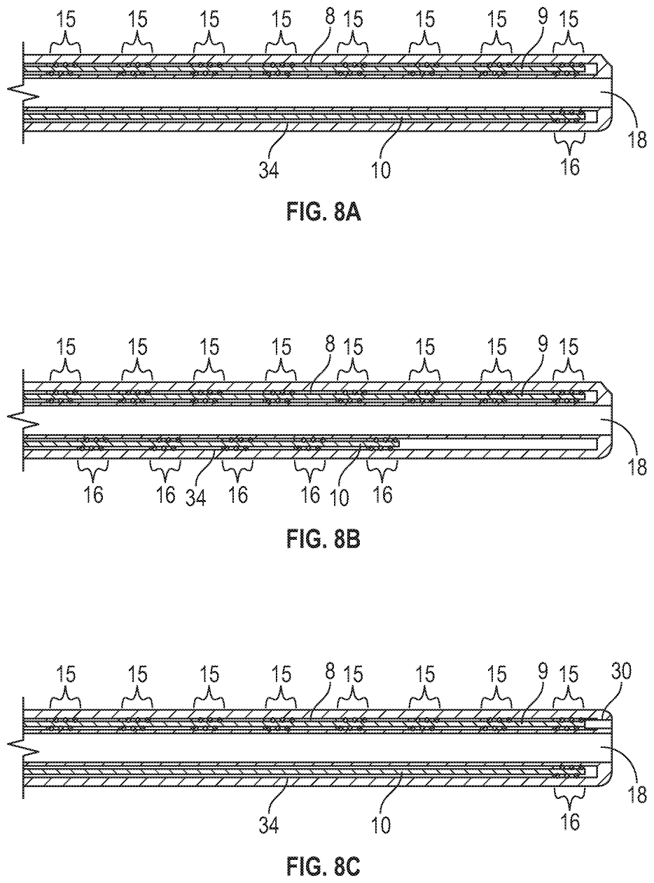

FIGS. 8a, 8b, 8c are schematic cross sectional drawings of exemplary bendable medical devices 3. This embodiment includes the first control wire 9 with multiple anchoring segments 15 which differ from the single anchor system detailed in the previous embodiments.

In FIG. 8a, the first control wire 9 includes multiple anchoring segments 15 along the longitudinal direction of the bendable body 7. On the opposing side, the second control wire 10 is deployed with an anchoring segment 16 at the distal end, wherein the actuation unit is coupled to the control wires 9 and 10, and can bend the bendable body 7 as directed. Since the first control wire 9 is fixed on the bendable body 7 at various points, thus limiting freedom of motion, the elastic property of the first control wire 9 is reflected as a resultant elastic property of the bendable body 7. Therefore, the first control wire 9 can modify the elastic property of the bendable body 7. The elastic property can account for bending stiffness, torsional stiffness, and axial rigidity. Specifically, in a case where the bendable body 7 is made of a soft material like elastomer, the first control wire 9 can prevent undesired contraction and expansion in the axial direction by increasing axial rigidity. Moreover, because the anchoring segments 15 are localized within the space of one lumen 34, this type of control wire 9 with multiple anchoring segments 15 can be deployed in multiple lumens 34 in the bending body 7, thus dramatically adding structural integrity to the bendable medical device 3 in a confined space.

In FIG. 8b, the second control wire 10 also includes multiple anchoring segments 16, and has the shorter length than the first control wire 9. With this configuration, the bendable body 7 can have segments with varying elastic properties along the longitudinal direction. In this particular embodiment, the proximal segment with the first control wire 9 and second control wire 10 has greater bending and torsional stiffness than the distal segment only with the first control wire 9, thus improving push-ability, and torque-ability of the bendable body 7 from the proximal end.

Moreover, the first control wire 9 and second control wire 10 can be made of a material visible in the medical imaging modality. In this particular embodiment, the first control wire 9 and second control wire 10 are radio-opaque and visible in X-ray fluoroscopy. Therefore, the clinical user can distinguish the shape of the first control wire 9 and second control wire 10 as the surrogate shape of the bendable body 7 under the X-ray fluoroscopy. Also, with the distal segment only containing the first control wire 9, the clinical user can distinguish the distal segment from the proximal segment in the X-ray fluoroscopy.

In FIG. 8c, the first control wire 9 may further act as a functional probe. In this particular embodiment, the first control wire 9 has an endoscope camera probe with the endoscopic camera unit 30 with illumination. With multiple anchoring segments 15, the endoscopic camera unit 30 and the harness cable for the endoscopic camera unit 30 are embedded in the wall 8. Specifically, since the endoscopic camera unit 30 is fixed to the bendable body 7 in all degrees of freedom of motion, the motion of the endoscopic camera unit 30 is consistent with the motion of the bendable body 7 during operation by the clinical user. This feature provides the user intuitive information for the operation as well as providing the system with consistent coordinate management between the bendable body 7 and the endoscopic camera unit 30.

In another embodiment, the first control wire 9 may contain an electromagnetic position tracking sensor or a shape sensing fiber, such that consistent coordinate management may be implemented, providing an advantage in the registration of the coordinates between the sensor system, the medical images, and the target/patient.

By having the control wire with a plurality of the anchoring segments, the bendable medical device can include the control wire fixed at all degrees of freedom of motions to the bendable body through the axial direction of the bendable body. This special control wire can maintain the geometrical relationship, i.e. a position and an orientation, between the control wire and the bendable body while the bendable body is bending, torsional and/or translational motions. Therefore, the control wire can effectively reflect its mechanical property to the resultant mechanical property of the bendable medical device with those constraints to modify the mechanical property of the bendable medical device. Specifically, an axial mechanical stiffness of the control wire can be reflected ideally while reducing risk of buckling of the control wire.

Moreover, the control wire can be utilized as a sensing element to localize the position and the orientation of the tip of the bendable body by leveraging consistency of the geometrical relationship to the bendable body.

Since the anchoring segments are localized within the space of one lumen 34, the bendable body 7 can have multiple different functional probes at the same time. For example, the bendable body 7 may include the electromagnetic position tracking sensor and the endoscopic camera unit, and can measure position and orientation of the endoscopic view in real-time.

Additional advantages may be recognized when having the first and the second bendable segments arranged a distance from the distal end of the bendable body, thus enhancing distal dexterity. The first and second bendable segments can be adjusted/manipulated independently to change the orientation of the distal end of the catheter as well as the position of the distal end three-dimensionally; both sections do not have to move in the same plane. With these motions, the distal end of the catheter can access a wide range of positions from a variety of orientations with the three bendable segments. Therefore, the bendable medical device provides physicians a wider addressable area and approaches for access of a medical instrument, such as biopsy forceps, a fine aspiration needle or an ablation probe.

Also, the third bendable segment can deform by external forces to follow the shape of tortuous pathways in the anatomy, such as lung airways, blood vessels and brain ventricles, while minimizing exerted force to the anatomy. Therefore, by following the shape of the anatomy, the third bendable segment can be navigated by the first and the second bendable sections when the catheter is inserted, and develop the delivery line for both the medial instruments for medical treatments and the driving force for control.

In addition, the bendable medical device provides consistent and accurate distal maneuvers by having the first and the second bendable segments. The second bendable segment detaches the motions of the first bendable segments from the rest of the proximal part of the bendable medical device. Because the proximal part of the catheter including the second bending bendable segment goes through the tortuous pathways in the anatomy, and interacts with the anatomy with many contacts along the pathways, those contacts interfere with the motion of the catheter and deteriorate control accuracy of the catheter. Moreover, this deterioration in itself is not systematic but random because the contact points and degree of contacts change by patient motion and the bendable medical device maneuvers. Therefore, by detaching the first and the second bendable segments, the bendable medical device can prevent the deterioration of control and achieves consistent and accurate distal maneuvers.

Finally, as the bendable body can avoid the uncontrollable contraction and the expansion in the bending motion by reflecting the axial stiffness of the first control wire, the bendable medical device can improve control accuracy of the bending, and reduce the cross talk among the different bending segments.

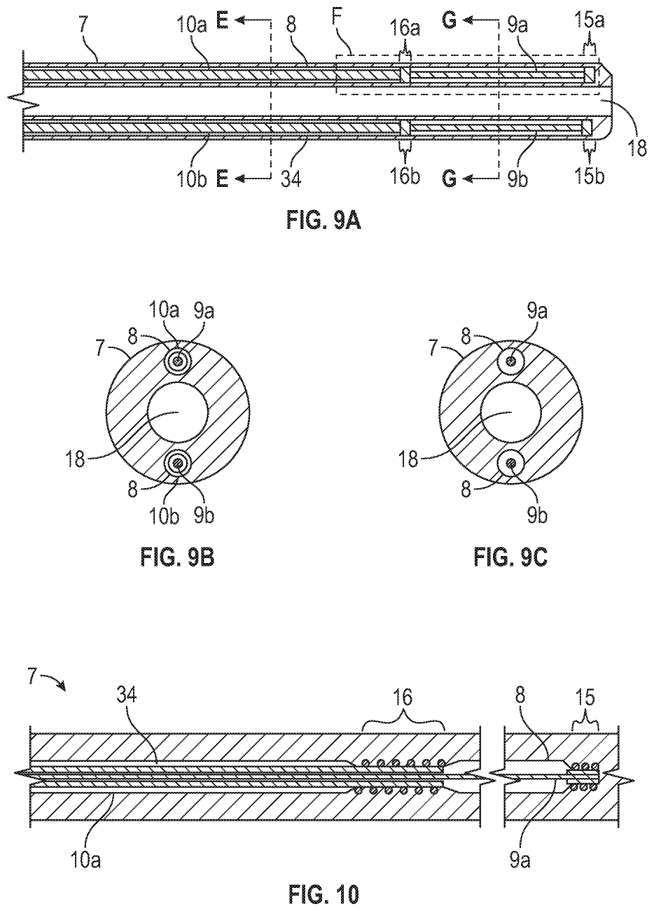

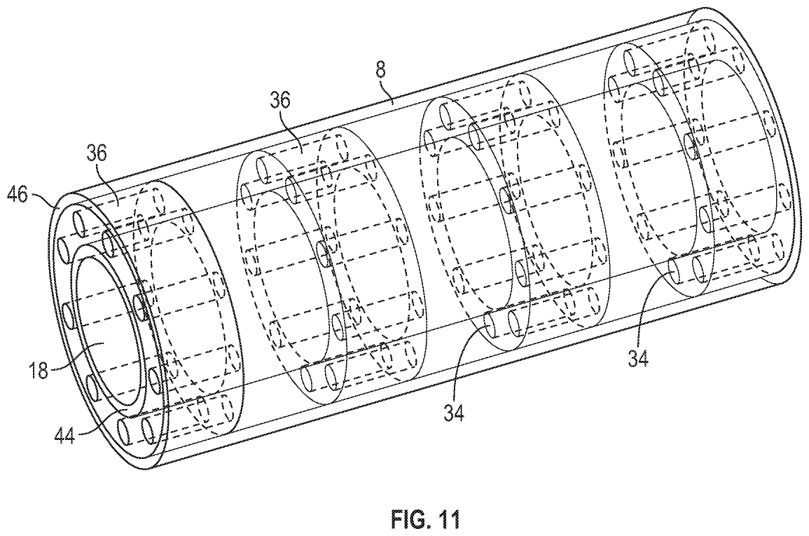

In yet another embodiment of the subject innovation, detailed in FIGS. 9a, 9b, 9c and FIG. 10, drawings depict an exemplary bendable medical device 3 featuring nested control wires. FIG. 9a is a schematic cross sectional drawing of the bendable medical device 3 including a center axis along longitudinal direction of the bendable body 7. FIG. 9b and FIG. 9c are schematic cut-away sectional views on lines E-E and G-G respectively. The first control wires 9a and 9b are nested in the second control wires 10a and 10b, respectively, while the first control wires 9a and 9b are slideable within the second control wires 10a and 10b. FIG. 10 is a close-up view of a box “F” in FIG. 9a. The second control wire 10 is configured in the same lumen 34 as the first control wire 9. Furthermore, the second anchoring segment 16 is longer than the first anchoring segment 15 to accommodate for the larger driving forces.

With this configuration, since the second control wire 10 shares the same axis and space with the first control wire 9, the second control wire 10 can cancel out the pushing and pulling forces of the first control wire 9 in the interval of the second control wire 10 without passing through other structures of the bendable body 7. For example, when the first control wire 9 is pulled to bend the distal end, the second control wire 10 can be pushed with the same magnitude of force to maintain the shape of the proximal part. Moreover, since the first control wire 9 is nested in the second control wire 10, the axes of those two control wires would keep consistency even when the bendable body 7 is bent along the pathways. Therefore, the bendable medical device 3 can effectively reduce the cross talk between different bendable segments during clinical operation. Especially when the bendable body involves relatively low axial stiffness and can distort its cross sectional plane from a plane without driving forces, the second control wire can compensate for those distortions in the interval of the second control wire by cancelling out the axial driving forces on the first control wire.

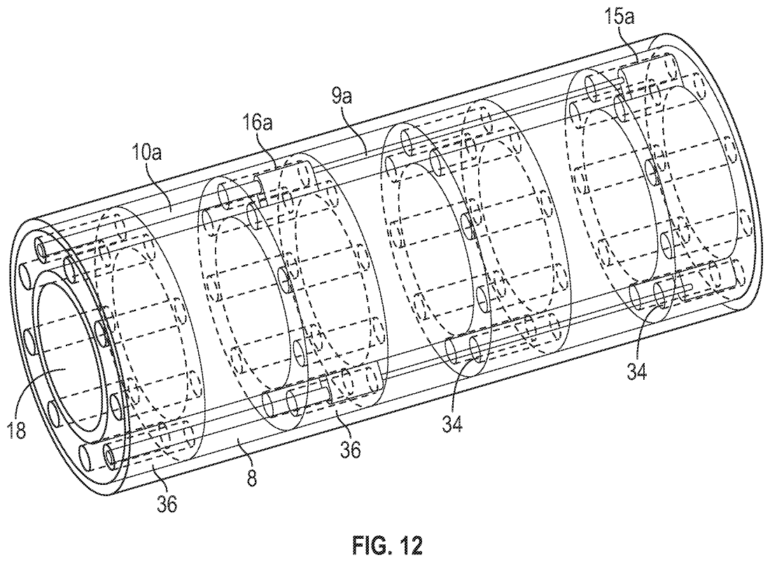

FIGS. 1i and 12 depict side perspective three-dimensional views of an exemplary bendable medical device, wherein the bendable medical device 3 includes at least two wire guides 36 confined within the inner diameter 40 and outer diameter 42 of the bendable body 7, wherein the wire guides 36 are configured a distance apart from one another and do not contact one another. The bendable body 7 comprises an inner lining 44 and an outer lining 46, which provides bendable support to the bendably body 7 while retaining the wire guides 36 in a constant position along the axial direction of the bendable body 7. Each wire guide 36 contains at least two lumens 34 for slideable housing of the control wires 9a-11c, and are configured to house the anchor segments (not shown) to be embedded into the wire guides 36. The space between adjacent wire guides, in cooperation with the resilient inner lining 44 and outer lining 46, allows the bendable body 7 to achieve a greater range of bending motion due to the open space between the wire guides 36.

In addition, this configuration of discrete sections and continuous outer lining 46 can be tuned to the required flexibility necessary for navigation to accommodate the anatomy of the patient. Furthermore, this embodiment allows the control wires 9 to be affixed to the bendable body 7 at the wire guides 36.

FIG. 12 further depict the use of a nested control wire 9 (as discussed in conjunction with FIGS. 9a-10) in conjunction with the at least two wire guides 36.

1. A medical apparatus comprising:
a bendable body comprising:
a hollow cavity extending the length of the bendable body; and
a wall formed about the hollow cavity;
at least one control wire slideably situated in the wall; and
an anchor configured at a distal end of the at least one control wire,
wherein, the anchor is affixed within the wall.

2. The apparatus of claim 1, wherein the anchor extends along an axial direction of the control wire with at least one projection element configured for embedding into the wall.

3. The apparatus of claim 2, wherein the projection element is selected from the group comprising of a bead, a disk, a leaf spring, a spiral coil, derivatives thereof, and combination therefrom.

4. The apparatus of claim 1, wherein the wall comprises at least one lumen for slideably accommodating the at least one control wire.

5. The apparatus of claim 1, further comprising a second control wire slideably situated in the wall and having an anchor for affixing the second control wire to the wall, wherein the position of the anchor for the first and the second control wires are different along the axial direction of the bendable body.

6. The apparatus of claim 1, wherein the at least one control wire includes a plurality of anchors, wherein the position of each anchor is different along the axial direction of the bendable body.

7. The apparatus of claim 1, further comprising a driving unit in communication with the at least one control wire, configured to actuate the at least one control wire in the wall.

8. The apparatus of claim 1, wherein the at least one control wire further comprises an outer wire and an inner wire, wherein the inner wire is slideably nested within the outer wire.

9. The apparatus of claim 8, wherein the outer wire has an anchor for affixing the outer wire to the wall and the inner wire has an anchor for affixing the inner wire to the wall, wherein the position of the anchor for the outer wire and the inner wires are different along the axial direction of the bendable body.

10. The apparatus of claim 1, wherein the control wire and anchor comprise of a radio opaque material.

11. The apparatus of claim 1, further comprising a functional probe selected from the group consisting of a position tracking sensor, a shape sensor, an endoscopic imaging probe, derivatives thereof, and combinations therefrom.

12. The apparatus of claim 11, wherein the functional probe is selected from the group consisting of the anchor, control wire, derivatives thereof, and combinations therefrom.

13. The apparatus of claim 1, wherein affixing the anchor to the wall comprises heating the wall and anchor to create a thermal fusion.

14. The apparatus of claim 1, wherein the wall comprises at least two wire guides, wherein the wire guides are spaced a distance from one another.

15. A medical apparatus prepared by a process comprising the steps of:
providing a bendable body comprising:
a hollow cavity extending the length of the bendable body;
a wall formed about the hollow cavity;
at least one control wire slideably situated in the wall; and
an anchor configured at a distal end of the at least one control wire;
heating the anchor configured in the wall to create a fusion between the wall and anchor; and
cooling the anchor configured in the wall to set the fusion.

16. A method for treating a subject, comprising:
providing a medical apparatus comprising:
a bendable body having a hollow cavity extending the length of the bendable body, and a wall formed about the hollow cavity;
at least one control wire slideably situated in the wall; and
an anchor configured at a distal end of the at least one control wire wherein the anchor is affixed to the wall;
advancing the medical apparatus into a subject;
bending the medical apparatus to accommodate obstacles in the subject; and
treating the subject once the medical apparatus advances to a desired target in the subject.

17. A medical apparatus comprising:
a bendable body comprising:
a hollow cavity extending the length of the bendable body; and
a plurality of wire guides spaced a distance from each other and extending the length of the hollow cavity;
at least one control wire slideably situated in at least one wire guide; and
an anchor configured at a distal end of the at least one control wire,
wherein, the anchor is affixed to the at least one wire guide.

18. The apparatus of claim 17, wherein the hollow cavity provides bendable support to the plurality of wire guides.

19. The apparatus of claim 17, wherein the anchor extends along an axial direction of the control wire with at least one projection element configured for embedding into the wire guide.

20. The apparatus of claim 17, wherein the wire guide comprises at least one lumen for slideably accommodating the at least one control wire.

21. The apparatus of claim 17, further comprising an outer lining for encompassing the apparatus.
